(19) United States
(12) Patent Application Publication
(10) Pub. No.: US 2024/0058399 A1
(43) Pub. Date: Feb. 22, 2024

(54) APPLICATION OF LACTOBACILLUS FERMENTUM IN PREPARING PRODUCTS FOR PREVENTING AND/OR TREATING THROMBUS

(71) Applicant: Thankcome Biological Science and Technology (Suzhou) Co., Ltd., Suzhou (CN)

(72) Inventors: Xin MA, Suzhou (CN); Yang YU, Suzhou (CN); Xueping YU, Suzhou (CN); Xin ZHAO, Suzhou (CN)

(21) Appl. No.: 18/498,630

(22) Filed: Oct. 31, 2023

Related U.S. Application Data
(63) Continuation of application No. PCT/CN2023/077307, filed on Feb. 21, 2023.

(30) Foreign Application Priority Data
Mar. 25, 2022 (CN) 202210306390.X

(51) Int. Cl. A61K 35/747; A61P 1/14; A61P 7/02; A61P 29/00; A61P 39/06; C12N 1/20

(52) U.S. Cl. CPC A61K 35/747; A61P 1/14; A61P 7/02; A61P 29/00; A61P 39/06; C12N 1/205; A61K 2035/115

(57) ABSTRACT

An application of a Lactobacillus fermentum in preparing products for preventing and/or treating thrombosis is provided in the present application, belonging to the technical field of microorganisms. The present application also discloses an inhibitory effect of a strain of Lactobacillus fermentum CQPC04 (LF-CQPC04) on thrombosis.

### CROSS-REFERENCE TO RELATED APPLICATIONS

This application is a continuation of PCT/CN2023/077307, filed Feb. 21, 2023 and claims priority of Chinese Patent Application No. 202210306390.X, filed on Mar. 25, 2022, the entire contents of which are incorporated herein by reference.

### INCORPORATION BY REFERENCE STATEMENT

This statement, made under Rules 77(b)(5)(ii) and any other applicable rule incorporates into the present specification of an XML file for a “Sequence Listing XML” (see Rule 831(a)), submitted via the USPTO patent electronic filing system or on one or more read-only optical discs (see Rule 1.52(e)(8)), identifying the names of each file, the date of creation of each file, and the size of each file in bytes as follows:
File name: sequence_347083_8059
Creation date: 10 Oct. 2023
Byte size: 20,277 bytes

### TECHNICAL FIELD

The present application relates to the technical field of microbiology, and in particular to an application of a Lactobacillus fermentum in preparing products for preventing and/or treating thrombus.

### BACKGROUND

Recently thrombus has become a very common disease in the elderly, second only to high blood pressure and heart disease, prompting research into antithrombotic foods and their active ingredients. Thrombus is a major cause of fatal cardiovascular disease, and chronic thrombus causes ischaemia, hypoxia and necrosis in the brain. However, most cardiovascular diseases have no obvious signs before they occur, with sudden and severe onset and a high risk of death if not treated promptly.

Inflammation is an important factor in triggering the initiation of thrombosis, and as inflammation increases it and often leads to more severe thrombus. The inflammatory response that occurs during the thrombus formation and development of thrombus is often accompanied by damage caused by oxidative stress and the production of large amounts of reactive oxygen species (ROS), a process that further exacerbates inflammation and increases the size of thrombus. Studies have shown that a healthy balance of intestinal flora facilitates the elimination of toxic substances from the body and prevents harmful substances produced by harmful bacteria from affecting the body's immunity, and that more beneficial bacteria in the body prevent many inflammatory diseases and inhibit the abnormal increase of free radicals in the body. Therefore, maintaining a healthy intestinal tract is an effective way to regulate inflammation and oxidative stress, and may also play a key role in preventing formation and development of thrombus.

As a naturally fermented vegetable, Sichuan pickles are rich in microorganisms, and the health benefits of some Sichuan pickles are likely to be largely related to the lactic acid flora they contain. Traditional Sichuan pickles contain a richer microbial composition than factory-produced pickles because of the different vegetables used in their preparation and differences in factors such as fermentation temperatures and fermentation duration. Some studies have confirmed that lactic acid bacteria isolated from Sichuan pickles are well colonized in the intestinal tract and have a good preventive and intervention effect on intestinal diseases including constipation and colitis. However, there are no reports on the role of lactic acid bacteria in reducing the risk of thrombosis.

### SUMMARY

It is an objective of the present application to provide an application of a Lactobacillus fermentum in preparing products for preventing and/or treating thrombus, so as to solve the problems existing in the prior art, and the Lactobacillus fermentum exerts a good inhibitory effect on thrombosis.

To achieve the above objective, the present application provides a following technical scheme:

an application of a Lactobacillus fermentum CQPC04 with a preservation number of CGMCC NO. 14493 in preparing products for preventing and/or treating thrombus.

Optionally, the Lactobacillus fermentum CQPC04 exerts an inhibitory effect on thrombosis by regulating intestinal microbial composition, increasing beneficial bacteria and maintaining intestinal health.

Optionally, the Lactobacillus fermentum CQPC04 improves coagulation abnormality caused by thrombus.

Optionally, the Lactobacillus fermentum CQPC04 reduces oxidative damage and inflammatory responses caused by thrombus.

The present application discloses the following technical effects:

the inhibitory effect of a strain of Lactobacillus fermentum CQPC04 (LF-CQPC04) on thrombosis is disclosed in the present application, with experimental results demonstrating that LF-CQPC04 is effective in modulating coagulation, serum and tissue levels of oxidative stress and inflammation; and studies on intestinal flora further suggest that LF-CQPC04 modulates the microbial composition of the intestinal tract, which may contribute to a healthy intestinal tract by increasing beneficial bacteria and thus exerting a good inhibitory effect on thrombus.

### BRIEF DESCRIPTION OF THE DRAWINGS

For a clearer illustration of the technical schemes in the embodiments of the present application or in the prior art, a brief description of the accompanying drawings to be used in the embodiments is given below. It is obvious that the accompanying drawings in the following description are only some embodiments of the present application and that other accompanying drawings are available to those of ordinary skill in the art without any creative effort.

FIG. 1 shows black tail morphologies of thrombosed mice.

FIG. 2A shows activated partial thromboplatin time (APTT) of thrombosed mice.

FIG. 2B shows thrombin time (TT) of thrombosed mice.

FIG. 2C shows fibrinogen (FIB) of thrombosed mice.

FIG. 2D shows prothrombin time (PT) of thrombosed mice.

FIG. 3A shows superoxidase dismutase (SOD) activities in serum of thrombosed mice.

FIG. 3B shows catalase (CAT) activities in serum of thrombosed mice.

FIG. 3C shows malondialdehyde (MDA) in serum of thrombosed mice.

FIG. 4A shows tumor necrosis factor-alpha (TNF-α) levels in serum of thrombosed mice.

FIG. 4B shows interleukin-6 (IL-6) levels in serum of thrombosed mice.

FIG. 4C shows nuclear factor kappa-B (NF-κB) levels in serum of thrombosed mice.

FIG. 4D shows interleukin-1 beta (IL-1β) levels in serum of thrombosed mice.

FIG. 5 shows hematoxylin-eosin (H-E) stained sections of tail tissues of thrombosed mice.

FIG. 6A shows mRNA expressions of Cu/Zn-SOD in colon tissues of thrombosed mice.

FIG. 6B shows mRNA expressions of Mn-SOD in colon tissues of thrombosed mice.

FIG. 6C shows mRNA expressions of CAT in colon tissues of thrombosed mice.

FIG. 6D shows mRNA expressions of NF-κB p65 in colon tissues of thrombosed mice.

FIG. 6E shows mRNA expressions of IL-6 in colon tissues of thrombosed mice.

FIG. 6F shows mRNA expressions of TNF-α in colon tissues of thrombosed mice.

FIG. 6G shows mRNA expressions of tumor necrosis factor-gamma (IFN-γ) in colon tissues of thrombosed mice.

FIG. 7A shows mRNA expressions of NF-κB p65 in tail vein tissues of thrombosed mice.

FIG. 7B shows mRNA expressions of intercellular cell adhesion molecule-1 (ICAM-1) in tail vein tissues of thrombosed mice.

FIG. 7C shows mRNA expressions of vascular cell adhesion molecule-1 (VCAM-1) in tail vein tissues of thrombosed mice.

FIG. 7D shows mRNA expressions of E-selectin in tail vein tissues of thrombosed mice.

FIG. 8 shows an average bacterial composition of microbiomes based on 16S rRNA diversity analysis in intestinal contents of thrombosed mice (on phylum level).

FIG. 9 shows an average bacterial composition of microbiomes based on 16S rRNA diversity analysis in intestinal contents of thrombosed mice (on genus level).

### DETAILED DESCRIPTION OF THE EMBODIMENTS

Various exemplary embodiments of the present application are now described in detail and this detailed description should not be considered as a limitation of the present application, but should be understood as a further detailed description of certain aspects, features and embodiments of the present application.

It is to be understood that the terms described in the present application are intended to describe particular embodiments only and are not intended to limit the present application. Further, with respect to the range of values in the present application, it is to be understood that each intermediate value between the upper and lower limits of the range is also specifically disclosed. Each smaller range between any stated value or intermediate value within a stated range and any other stated value or intermediate value within a stated range is also included in the present application. The upper and lower limits of these smaller ranges may be independently included or excluded from the scope.

Unless otherwise stated, all technical and scientific terms used herein have the same meaning as commonly understood by those of ordinary skill in the field described in the present application. Although the present application describes only preferred methods and materials, any methods and materials similar or equivalent to those described herein may also be used in the implementation or testing of the present application. All literature referred to in this specification is incorporated by reference for the purpose of disclosing and describing the methods and/or materials associated with said literature. In the event of conflict with any incorporated literature, the contents of this specification shall prevail.

Without departing from the scope or spirit of the present application, various improvements and variations may be made to specific embodiments of the specification of the present application, as will be apparent to those skilled in the art. Other embodiments derived from the specification of the present application are obvious to the skilled person. The specification and embodiments of the present application are exemplary only.

As used herein, the terms “contain”, “comprise”, “include”, “have”, etc. are open-ended terms, i.e. meaning including but not limited to.

### Embodiment 1

1 Materials and Methods

1.1 Materials and Reagents

The Lactobacillus fermentum CQPC04 (LFCQPC04) is isolated and identified from pickles, and has been conserved in the China General Microbiological Culture Collection Center (CGMCC for short, with an address of No. 3, Yard No. 1 West Beichen Road, Chaoyang District, Beijing), under a preservation number of CGMCC No. 14493. The place of collection was Chongqing, China.

Male Kunming mice of specific pathogen free (SPF) grade, 6 weeks old, weighing 23±2 grams (g), are purchased from the Experimental Animal Center of Chongqing Medical University (production license number: SCXK(YU)2018-0003). The experiments in this study are approved by the Animal Experiment Ethics Committee of Chongqing Collaborative Innovation Center for Functional Foods (approval number: 2021070010B).

Heparin: SIGMA Company, USA; detection kits of tumor necrosis factor-α (TNF-α), interleukin-6 (IL-6), nuclear factor kappa-B (NF-κB): Shanghai Enzyme-linked Biotechnology Co., Ltd.; superoxidase dismutase (SOD), catalase (CAT), and malondialdehyde (MDA): Nanjing Jiancheng Bioengineering Institute; TRIzol reagent: Invitrogen Company, USA; SYBR Green PCR Master Mix and qPCR primers: Thermo Fisher Scientific, USA; other reagents are Chinese analytical reagents.

1.2 Instruments and Equipment

PUN-2048A semi-automatic coagulometer: Beijing Perlong Co., Ltd.; BX43 microscope: Olympus Corporation of Japan; Varioskan LUX multimode microplate reader and SteponePlus Quantitative PCR Instrument: Thermo Fisher Scientific, USA; Agilent 2100 Bioanalyzer: Agilent Technologies, Inc., USA.

1.3 Methods

1.3.1 Animal Experiment

The experiment starts after 7 days (d) of adaptive feeding of Kunming mice. Mice weighing 23±2 g are selected after adaptive feeding. The selected mice are randomly grouped into the normal group, model group, heparin group, LF-CQPC04 low concentration (LF-CQPC04-L) group and LF-CQPC04 high concentration (LF-CQPC04-H) group, 10 mice in each group, total 50 mice. Mice in the normal group are injected intraperitoneally with saline (0.01 milliliter per gram body weight for 1 d, mL/g bw·d) and mice in all other groups are injected intraperitoneally with carrageenan solution (0.2%, 0.01 mL/g bw·d) to induce thrombosis for 10 d. The mice are subjected to gavage with heparin solution (20 milligrams per gram body weight for 1 d, mL/g bw·d) in the heparin group, and LF-CQPC04 in the LF-CQPC04-L group (10⁸ colony-forming units per kilogram body weight for 1 d, CFU/kg bw·d) and LF-CQPC04-H group (10⁹ CFU/kg bw·d), respectively. After the 10-d experiment, the mice are executed by cervical dislocation, photographed to determine the length of the black tail, then dissected to remove the contents of the colon, while the heart blood and colon are taken from the mice for later use.

1.3.2 Determination of Blood Coagulation

The collected mice blood is placed in a centrifuge tube, and then the activated partial thromboplatin time (APTT), thrombin time (TT), fibrinogen (FIB) and prothrombin time (PT) of mice blood are measured by a semi-automatic coagulometer.

1.3.3 Determination of Oxidation Index

The collected mice blood is centrifuged at 4 degrees Celsius (° C.) for 10 minutes (min) at 4000 revolutions per minute (rpm) to obtain supernatant serum, and then the SOD, CAT activity and MDA levels in the mice serum are determined by detection kits.

1.3.4 Determination of Inflammatory Cytokines

The serum of mice is prepared according to the 1.3.3, and then the levels of TNF-α, IL-6, NF-κB and IL-1β in the serum of mice are determined by cytokine detection kit.

1.3.5 Pathological Observation

Tail tissues of dissected mice are fixed in 10% formalin solution, followed by dehydration for 48 h. The tissues are then embedded in paraffin and sectioned, and stained with hematoxylin-eosin (H&E). Finally, the pathological changes in the gastric tissues are observed under an optical microscope.

1.3.6 Experiment of Quantitative Polymerase Chain Reaction (qPCR)

The tissue from the middle section of mouse colon and the stripped tissue from the tail vein of mouse are taken out 100 mg respectively, the tissues are washed with normal saline and then added to clean saline in the ratio of 1:9. After the tissues are homogenized, RNAzol reagent (1.0 mL) is added to extract the RNA from the mice tissues, then the concentration of the extracted RNA is adjusted to 1 microgram per microliter (μg/μL). Then reverse transcription is carried out to obtain cDNA, followed by the preparation of the reaction system, which consists of cDNA (1 μL), SYBR Green PCR Master Mix (10 μL), sterile distilled water (7 μL), and PCR primers (1 μL each for upstream and downstream, at a concentration of 10 micromole per liter (μmol/L)). The prepared reaction solutions are amplified under the following amplification conditions: 95° C. sustained for 60 seconds (s); 95° C. sustained for 15 s for 40 cycles; 55° C. sustained for 30 s; 72° C. sustained for 35 s; 95° C. sustained for 30 s; and 55° C. sustained for 35 s. Glyceraldehyde-3-phosphate dehydrogenase (GAPDH) is used as an internal reference gene (Table 1) and the relative amounts of each expressed gene are analysed using the 2^{-ΔΔct} method.

TABLE 1
Primer sequences used in this experiment

| Expressions | Sequences of upstream primers | Sequences of downstream primers |
|---|---|---|
| Cu/Zn-SOD | 5′- | 5′- |
| | AACCAGTTGTGTTGTGAGGAC-3′ | CCACCATGTTTCTTAGAGTGAGG-3′ |
| | (SEQ ID NO: 1) | (SEQ ID NO: 2) |
| Mn-SOD | 5′- | 5′-CTCGGTGGCGTTGAGATTGTT-3′ |
| | CAGACCTGCCTTACGACTATGG- | (SEQ ID NO: 4) |
| | 3′ (SEQ ID NO: 3) | |
| CAT | 5′-GGAGGCGGGAACCCAATAG-3′ | 5′-GTGTGCCATCTCGTCAGTGAA-3′ |
| | (SEQ ID NO: 5) | (SEQ ID NO: 6) |
| NF-κB p65 | 5′-GAGGCACGAGGCTCCTTTTCT- | 5′- |
| | 3′ (SEQ ID NO: 7) | GTAGCTGCATGGAGACTCGAACA- |
| | | 3′ (SEQ ID NO: 8) |
| ICAM-1 | 5′-TCCGCTACCATCACCGTGTAT-3′ | 5′-TAGCCAGCACCGTGAATGTG-3′ |
| | (SEQ ID NO: 9) | (SEQ ID NO: 10) |
| VCAM-1 | 5′-TTGGGAGCCTCAACGGTACT-3′ | 5′-GCAATCGTTTTGTATTCAGGGGA-3′ |
| | (SEQ ID NO: 11) | (SEQ ID NO: 12) |
| E-selectin | 5′-ATAACGAGACGCCATCATGC-3′ | 5′-TGTCCACTGCCCTTGTGC-3′ (SEQ |
| | (SEQ ID NO: 13) | ID NO: 14) |
| IL-6 | 5′-ATGAAGTTCCTCTCTGCAA-3′ | 5′-AGTGGTATCCTCTGTGAAG-3′ |
| | (SEQ ID NO: 15) | (SEQ ID NO: 16) |
| TNF-α | 5′-ATGGGGGGCTTCCAGAA-3′ | 5′-CCTTTGGGGACCGATCA-3′ (SEQ |
| | (SEQ ID NO: 17) | ID NO: 18) |
| IFN-γ | 5′-GCTTTGCAGCTCTTCCTCAT-3′ | 5′-GTCACCATCCTTTTGCCAGT-3′ |
| | (SEQ ID NO: 19) | (SEQ ID NO: 20) |
| GAPDH | 5′- | 5′-CTTCCCATTCTCGGCCTTG-3′ |
| | TGACCTCAACTACATGGTCTACA- | (SEQ ID NO: 22) |
| | 3′ (SEQ ID NO: 21) | |

1.3.7 High-Throughput Sequencing

The intestinal contents of mice are purified by AMPure XP magnetic beads and the free primers and primer dimers in the amplification products are removed. Advancement and library construction are performed on samples to be sequenced using a universal Illumina adapter and index. The DNA concentration of each PCR product is determined prior to sequencing using the Qubit 2.0 Green double-stranded DNA assay and quality controlled using a bioanalyser. Depending on the concentration of the amplicons, they are combined in equimolar ratios and sequenced using the Illumina MiSeq system according to the manufacturer's instructions. The data are finally analysed using the online Majorbio cloud platform.

1.4 Statistical Analysis

The data obtained by completing three parallel experiments are expressed as mean±standard deviation. One-way analysis of variance is then used to analyse the significant differences (P<0.05) among the data.

2 Results and Analysis

2.1 Mouse Black Tail Length

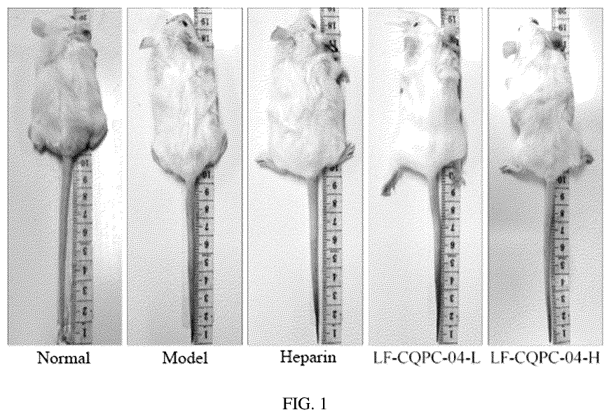

After the mice are injected intraperitoneally with carrageenan, a black area gradually appears at the tip of the tail in all groups except the normal group, suggesting the formation of thrombus in the tail leading to a black tail (FIG. 1). The black tail of the mice in the model group is the longest (9.4±0.4 centimeters (cm)), significantly higher than that of the other groups (P<0.05). The black tail length of mice in the LF-CQPC04-H group (3.2±0.4 cm) is similar to that of the heparin group (3.0±0.2 cm) with no significant difference. In contrast, the black tail length in the LF-CQPC04-H group and the heparin group is significantly shorter than that in the LF-CQPC03-L group (7.8±0.5 cm, P<0.05).

2.2 APTT, TT, FIB and PT Levels of Mice

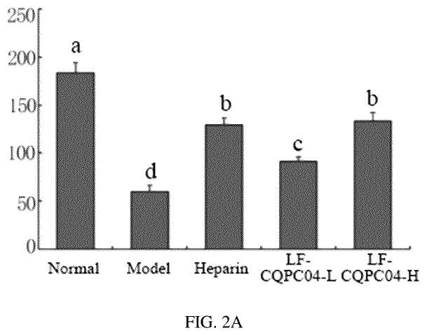

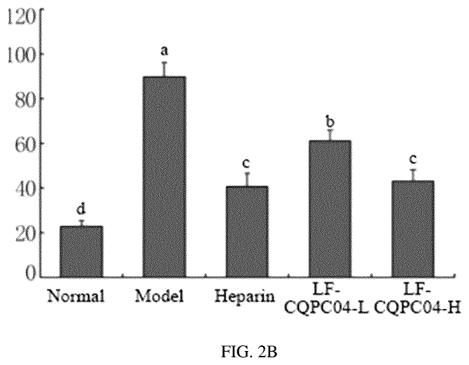

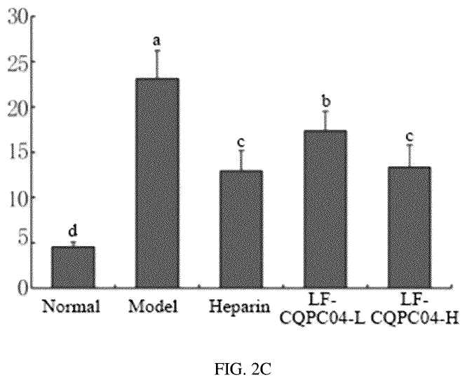

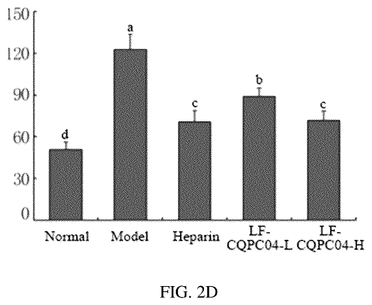

The APTT level of the normal group is significantly higher than those of the other groups (FIGS. 2A-2D), while the TT, FIB and PT levels are significantly lower than those of the other groups (P<0.05). In contrast, the APTT level of the model group is significantly lower than those of the other groups, while TT, FIB and PT levels are significantly higher than those of the other groups (P<0.05). The APTT level in the LF-CQPC04-H group is significantly higher than that in the LF-CQPC04-L group; TT, FIB and PT levels are significantly (P<0.05) lower than those in the LF-CQPC04-L group. The APTT, TT, FIB and PT levels in the LF-CQPC04-H group are similar to those in the heparin group and the differences are not statistically significant (P>0.05).

2.3 SOD, CAT Activities and MDA Level in Serum of Mice

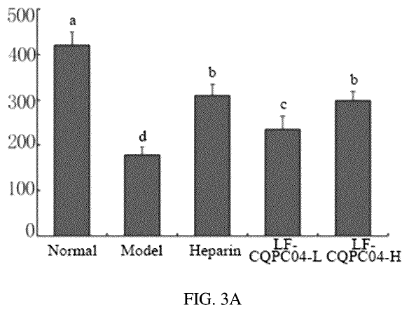

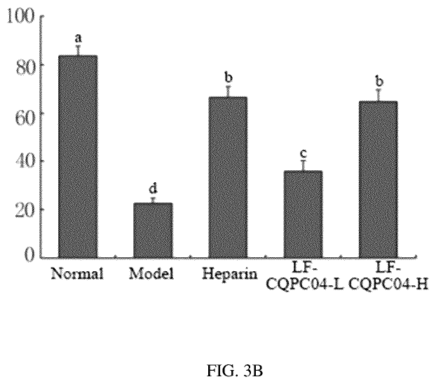

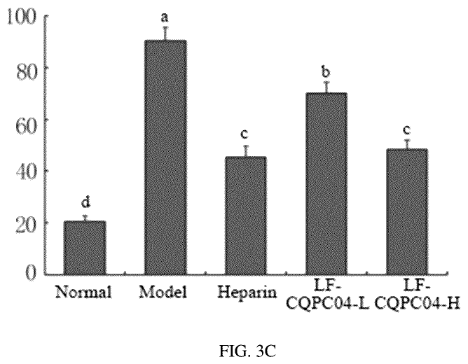

The results of serum test show that the activities of SOD and CAT in serum of normal mice are the highest (P<0.05) and the level of MDA is the lowest (FIGS. 3A-3C). The SOD and CAT activities in the heparin group and LF-CQPC04-H group are slightly lower than those in the normal group and higher than those in the LF-CQPC04-L group (P<0.05). The SOD and CAT activities of mice in the model group are the lowest (P<0.05). MDA level in the model group is the highest, and MDA level in the LF-CQPC04-L group is lower than that in the model group and higher than that in the heparin and LF-CQPC04-H groups (P<0.05).

2.4 Levels of TNF-α, IL-6, NF-κB and IL-1β in Serum of Mice

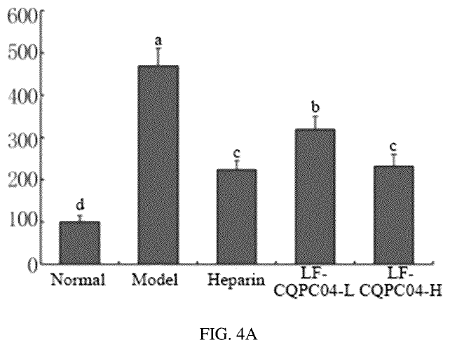

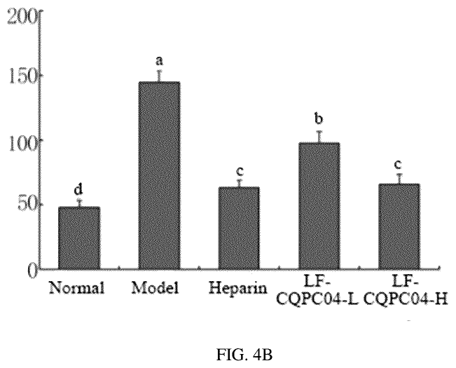

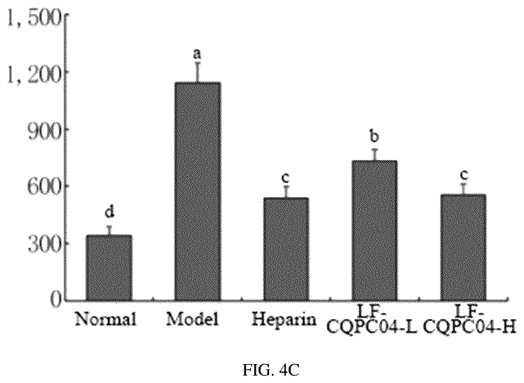

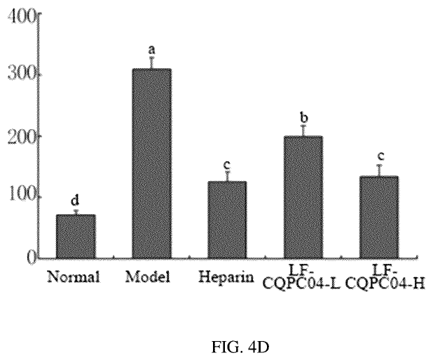

The experimental results show that the levels of TNF-α, IL-6, NF-κB and IL-1β in serums of normal group, heparin group, LF-CQPC04-H group, LF-CQPC04-L group and model group show a trend from low to high (FIGS. 4A-4D). Among them, TNF-α, IL-6, NF-κB and IL-1β levels in the heparin group and LF-CQPC04-H group are significantly lower than those in the LF-CQPC04-L group (P<0.05), but the differences between the heparin and LF-CQPC04-H groups are not statistically significant (P>0.05).

2.5 Pathological Observation of Mice Tail Tissues

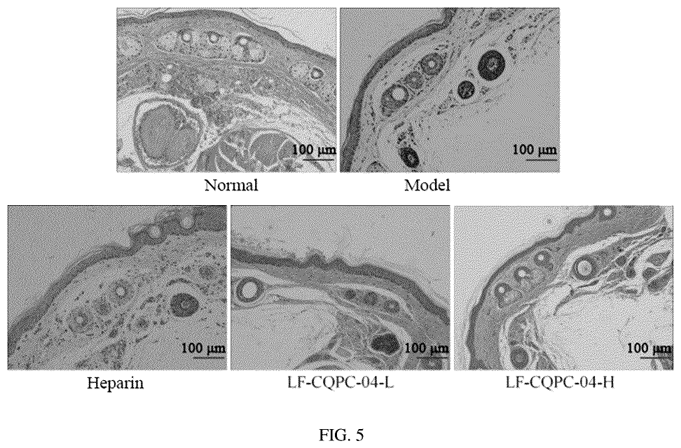

H&E stained sections show that the tail vessels of mice in the normal group are round and clean with smooth vessel walls (FIG. 5) Inflammatory exudation, haemorrhagic lesions, platelet aggregation and intravascular thrombosis in the tail vessels are observed in the model group. Both LF-CQPC04 and heparin groups show reduction of the lesions in the tail vessels of mice. LF-CQPC04-H and heparin groups show better effects than LF-CQPC04-L group and the effects of LF-CQPC04-H group and heparin group are similar.

2.6 Expressions of Related mRNA in Colon Tissues of Mice

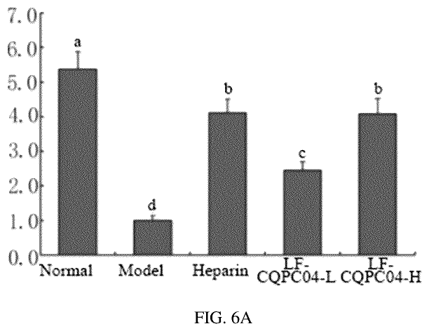

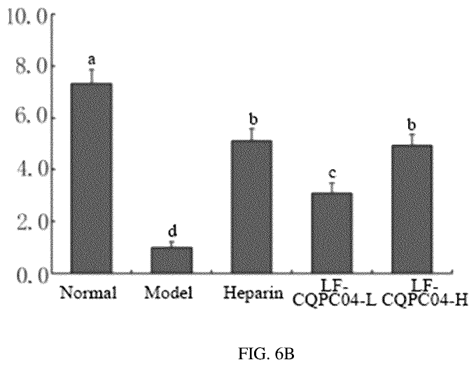

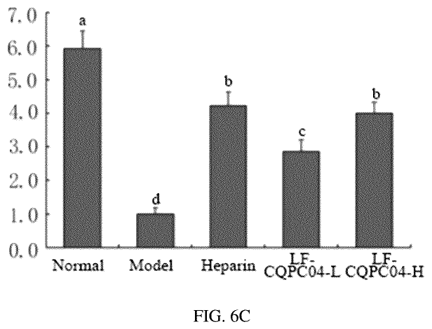

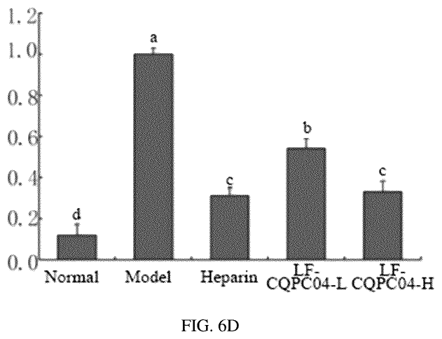

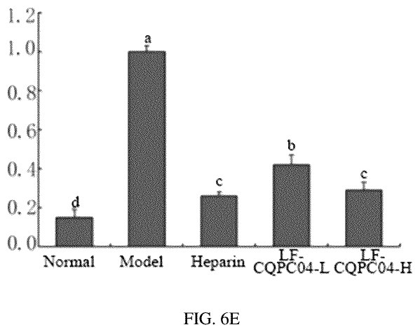

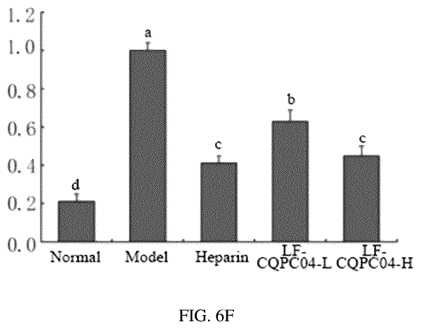

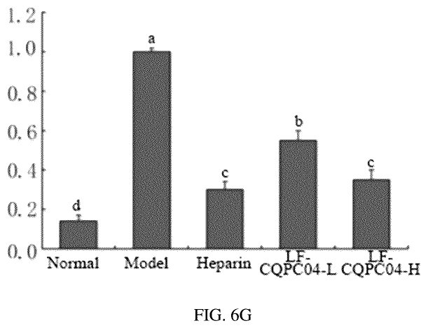

The results of qPCR show that the mRNA expressions of Cu/Zn-SOD, Mn-SOD and CAT are the strongest in the colon tissues of normal mice (FIGS. 6A-6G), but the lowest in the model group. The expression intensity of Cu/Zn-SOD, Mn-SOD and CAT in colon tissues of mice in LF-CQPC04-H group and heparin group is similar, and stronger than that in LF-CQPC04-L group. The expressions of NF-κB p65, IL-6, TNF-α and IFN-γ in the colon tissues of normal mice are significantly lower than those in other groups, while those in model group are significantly higher than those in other groups (P<0.05). Both LF-CQPC04 group and heparin group show down-regulated expressions of NF-κB p65, IL-6, TNF-α and IFN-γ in the colon tissues of thrombosed mice. The effects of LF-CQPC04-H group are similar to those of heparin group, and both of them are significantly better than those of LF-CQPC04-L group (P<0.05).

2.7 mRNA Expressions in Vein Tissues of Mice Tails

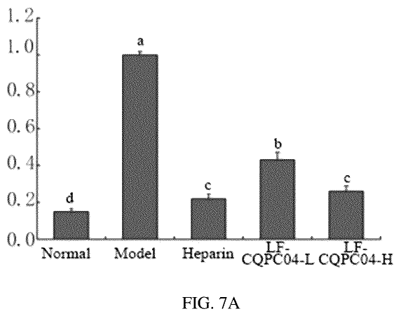

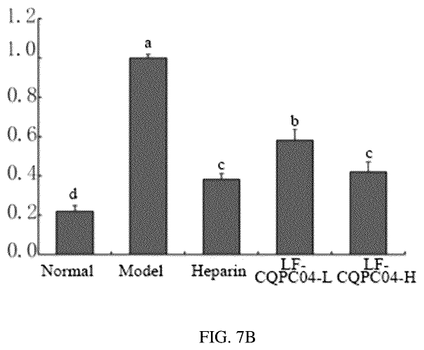

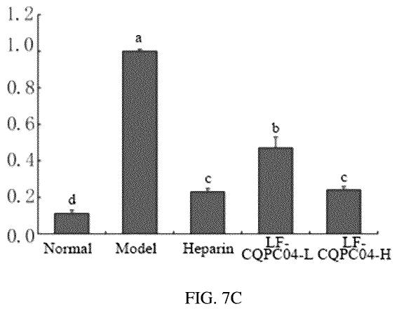

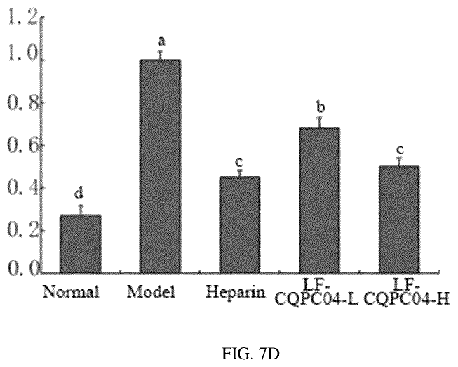

The results of qPCR experiment show that the mRNA expressions of NF-κB p65, intercellular cell adhesion molecule-1 (ICAM-1), vascular cell adhesion molecule-1 (VCAM-1) and E-selectin in the tail vein of mice in the model group are the strongest (FIGS. 7A-7D). LF-CQPC04-L group, LF-CQPC04-H group and heparin group show significantly (P<0.05) down-regulated expression of NF-κB p65, ICAM-1, VCAM-1 and E-selectin in the tail vein of thrombosed mice. At the same time, these expressions are not significantly different between mice in the LF-CQPC04-H and heparin groups (P>0.05) and are only slightly higher than those in the normal group.

2.8 Microbial Diversity of Intestinal Contents of Mice

By analyzing the Alpha diversity index, the information of the richness and diversity of the flora in the intestinal contents of mice is obtained, with results as shown in Table 2, where significant differences exist among the values of ACE, Chao, Shannon index and Simpson among the groups (P<0.05). Compared with the model group, the flora abundance in the feces of mice in normal group, LF-CQPC04 group and heparin group is higher, and there are great differences among different doses of LF-CQPC04 group, with the diversity index of flora in LF-CQPC04-H group being higher than that in LF-CQPC04-L group. Moreover, Alpha diversity analysis also shows that the diversity of intestinal flora in mice after intragastric administration of LF-CQPC04 is significantly higher than that in the model group, with the abundance of fecal flora in LF-CQPC04-H group being the highest, higher than that in normal group and heparin group.

TABLE 2
Alpha diversity index

| Groups | Sobs | Ace | Chao | Shannon | Simpson | Coverage |
|---|---|---|---|---|---|---|
| Normal group | 722 | 760.773959 | 750.8 | 4.489651 | 0.031806 | 0.999413 |
| Model group | 112 | 122.870342 | 120.052632 | 1.31621 | 0.435136 | 0.999835 |
| Heparin group | 553 | 574.119988 | 573.204082 | 4.129883 | 0.034128 | 0.999587 |
| LF-CQPC04-L | 720 | 766.191475 | 750.348315 | 4.484631 | 0.026678 | 0.999321 |
| LF-CQPC04-H | 803 | 861.724775 | 836.074074 | 4.495502 | 0.027374 | 0.999128 |

2.9 Microbial Composition of Intestinal Contents in Mice

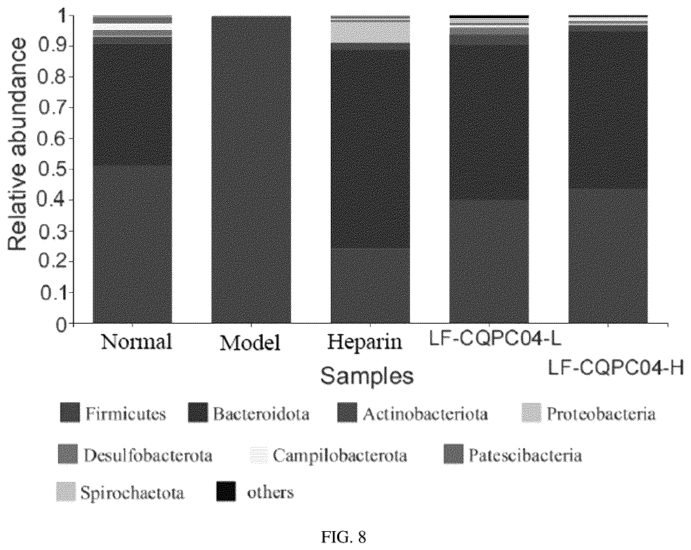

Based on the results of the taxonomic analysis of microorganisms, it is possible to observe the structural composition of the community in the intestinal contents of the different groups of mice at each taxonomic level. As shown in FIG. 8, the community composition of the five groups is dominated at the phylum level by three phyla, namely Bacteroidetes, Firmicutes and Actinobacteria. The proportion of the Firmicutes to the Bacteroidetes is significantly lower (1.28) in the normal group compared to that of the model group. After the intervention of LF-CQPC04, the proportion of Firmicutes to Bacteroidetes shows significant decreasing compared with those of the model group and the normal group, with LF-CQPC04-L group: 0.82 and LF-CQPC04-H group: 0.78. The positive control heparin group is 0.40, with significant differences between the groups. The proportion of Firmicutes to Bacteroidetes decreases with the increase of LF-CQPC04 dosage.

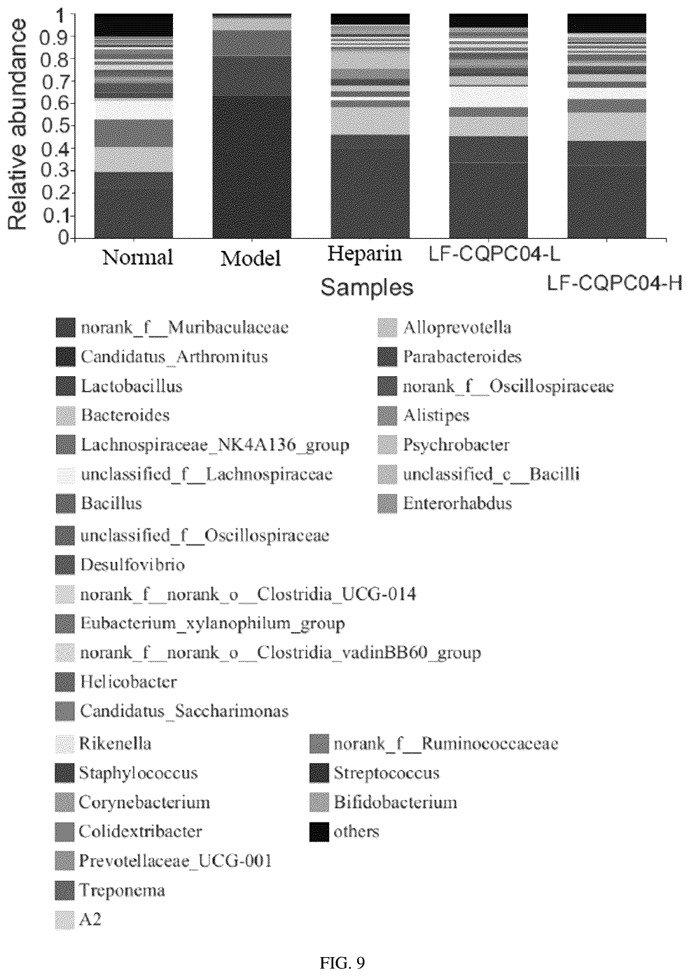

FIG. 9 shows the floristic composition of five taxa at the genus level. The main flora in the model group are Parabacillus, Klebsiella and Bacteroides. The flora in the normal group includes Bacteroides, Lactobacillus, and the flora in the LP-CQPC04-H group includes Bacteroides, Lactobacillus, Alistipes and Lachnospiraceae. After oral administration of LF-CQPC04, the content of lactic acid bacteria in the intestine of mice shows significant increase (P<0.05). The LP-CQPC04-H group shows the most significant increasing effect, close to that of the normal group. In addition, the quantity of pathogenic bacteria such as Klebsiella is reduced after oral administration of LP-CQPC04 compared to that of the model group and tends to decrease as the dose of LP-CQPC04 increases.

Carrageenan is capable of causing the inflammation-related thrombosis in the tail vessels of mice, resulting in a mixed thrombus filling the tail vessels, which in turn leads to ischaemic necrosis of the tail tissue and is visible as black to the naked eye in the tail. The length of the black tail in mice is therefore an important experimental indicator to visually determine the severity of thrombosis. It is also demonstrated that carrageenan contributes to the formation of a distinct black tail in the tail of mice. Both heparin and LF-CQPC034 groups show reduced blackening of the tail caused by thrombus, and the effectiveness of high concentrations of LF-CQPC04 is better than that of low concentrations of LF-CQPC04. In addition, LF-CQPC03-H provides a similar effect to that of the commonly used antithrombotic drug heparin.

In the process of thrombosis, a large amount of coagulation factors is consumed, resulting in a prolongation of the PT, while the loss of coagulation factors leads to a shortening of the APTT. Meanwhile, the continuous conversion of FIB to fibrin by thrombin, a major component of the thrombus, contributes to the hypercoagulable state of the blood. As the blood remains in such a state of hypercoagulation, and as the fibrin content of the blood continues to increase, the body is forced to enhance fibrinolysis to degrade the fibrin, thus prolonging the TT. In this experiment, both LF-CQPC04 and heparin are capable of regulating APTT, TT, FIB and PT, in particular, LF-CQPC04-H and heparin are capable of bringing these indicators closer to normal and improving the coagulation abnormalities caused by thrombus in mice.

Free radical accumulation is an important factor in triggering and exacerbating thrombosis, with reactive oxygen species (ROS) directly activating platelets and increasing the risk of thrombosis; ROS also activates the NF-κB signaling pathway, promoting the secretion of thrombotic molecules and leading to the formation of venous thrombus. Studies have shown that free radical scavengers prevent iron ion-induced thrombosis, indicating the important role of oxidative stress in thrombosis. SOD catalyzes the dismutation of superoxide anion radicals to produce oxygen and hydrogen peroxide, and plays a crucial role in balancing oxidation and antioxidant activity in the body, with two important types of SOD found in mammals being Cu/Zn-SOD and Mn-SOD. CAT is an enzyme scavenger that promotes the breakdown of hydrogen peroxide into molecular oxygen and water, and the enzymatic activity provides the body with an antioxidant defence. Being antioxidant enzymes, SOD and CAT are both important enzymes in preventing damage to the body caused by ROS and are also effective active substances in inhibiting thrombosis caused by oxidative stress. In living organisms, free radicals cause lipid peroxidation, resulting in the formation of MDA, which indirectly reflects the extent of oxidative stress damage to the body, and therefore MDA level may also be an important indicator of thrombosis. In this experiment, the level of oxidative stress increases in mice after thrombus is formed, the level of SOD and CAT antioxidant enzymes and mRNA expression decrease, and the level of MDA increases. The results are the same as previous studies, and such results indicate that thrombosis is closely related to oxidative stress. At high concentrations, LF-CQPC04 promotes these oxidation-related indicators reaching near-normal levels, and the effect of LF-CQPC04-H reaches the level of that of the antithrombotic drug heparin.

Inflammation has a mutually reinforcing circulatory effect in the process of thrombosis. After inflammation occurs in the body, inflammatory mediators such as TNF-α, IL-6, NF-κB and IL-1β are produced in large quantities, among which TNF-α regulates the downstream NF-κB signaling pathway, and then promotes the release of inflammatory factors such as IL-6, IL-1β and IFN-γ from macrophages, thereby exacerbating vascular endothelial cell damage and contributing to the gradual formation of thrombus. The cytokine levels or mRNA expression of TNF-α, IL-6, NF-κB, IL-1β and IFN-γ also exhibit a substantial increase after thrombosis in this experiment of mice. LF-CQPC04 exhibits a significant cytokine inhibitory effect and the effect of LF-CQPC04-H is comparable to that of the drug heparin. Inflammation and oxidative stress in mice lead to thrombus in the tail and also cause intestinal inflammation leading to intestinal endothelial cell damage. Therefore, observation of the severity of intestinal damage is also a way to determine the degree of experimental thrombosis induced by carrageenan. Observations in this experiment indicate that the expression of inflammation in mouse colonic tissue also changes after thrombosis in the tail, again demonstrating that tail thrombosis in mice is closely related to intestinal lesions. Both the drug heparin and LF-CQPC04 suppress the activation and enhancement of these expression changes. Thus, LF-CQPC04, with probiotic potential, is capable of acting in a similar way to heparin.

NF-κB plays an influential role in the initiation and development of thrombosis as a key factor in inflammation, promoting the accumulation of platelets and increasing inflammation, disrupting the coagulation homeostasis in the body and triggering the initiation of thrombosis. ICAM-1 is essential in the regulation of cell matrix adhesion, while VCAM-1 further promotes platelet accumulation. Activation and overexpression of both ICAM-1 and VCAM-1 exacerbate inflammation and induce thrombosis. Activation of E-selectin exacerbates endothelial cell verification, causing damage to endothelial cells and affecting their permeability, as well as affecting the total leukocyte count in the blood and regulating the adhesion between vascular endothelial cells, all of which are directly related to thrombosis. NF-κB, on the other hand, is a key regulator of ICAM-1, VCAM-1 and E-selectin, thereby associating the expression of related genes and influencing the initiation of thrombosis. The present experiment also reveals that LF-CQPC04 has a significant regulatory effect on the expression of NF-κB, ICAM-1, VCAM-1 and E-selectin, thereby regulating the development of thrombus.

Studies have shown that intestinal flora is closely related to inflammation and oxidative stress, and clinical data suggest that the intestinal flora of patients with disease differs significantly from that of healthy people, with an increase in harmful bacteria in the intestinal flora of patients with disease and a greater change in the abundance of flora compared to the normal state. The imbalance of intestinal flora is closely related to cardiovascular disease, and significant changes in intestinal flora are seen in the pathogenesis of hyperlipidaemia, obesity and type 2 diabetes. In the present experiment, thrombus causes changes in the abundance of intestinal flora in mice, and LF-CQPC04 regulates bacterial abundance and restores intestinal health in thrombosed mice, suggesting a possible role in inhibiting thrombosis.

Parabacteroides are microorganisms associated with the promotion of obesity; Klebsiella are the second most harmful bacteria in the intestine after Escherichia coli. The results of the experiment show that the model mice have more harmful bacteria in their intestines, including Parabacteroides and Klebsiella. Lactobacillus is a group of microorganisms that can be used as probiotics, and normal mice have a high concentration of beneficial Lactobacillus in their intestines. Alistipes has been proved to interfere with inflammation, while Lachnospiraceae has been demonstrated to have a protective effect on the haematopoietic and intestinal systems. Lactobacillus, Alistipes and Lachnospiraceae are all important beneficial bacteria in the human body and LF-CQPC04 is capable of increasing the abundance of these beneficial bacteria in the intestine of thrombosed mice, thereby restoring intestinal health and reducing inflammation, which in turn inhibits thrombosis.

The above-mentioned embodiments only describe the preferred mode of the present application, and do not limit the scope of the present application. Under the premise of not departing from the design spirit of the present application, various modifications and improvements made by ordinary technicians in the field to the technical scheme of the present application shall fall within the protection scope determined by the claims of the present application.

What is claimed is:

1. An application of a Lactobacillus fermentum CQPC04 with a preservation number of CGMCC NO. 14493 in preparing products for treating thrombus, wherein the thrombus is inflammation-related thrombus.

2. The application of the Lactobacillus fermentum CQPC04 in preparing products for treating thrombus according to claim 1, wherein the Lactobacillus fermentum CQPC04 exerts an inhibitory effect on thrombosis by regulating intestinal microbial composition, increasing beneficial bacteria and maintaining intestinal health.

3. The application of the Lactobacillus fermentum CQPC04 in preparing products for treating thrombus according to claim 1, wherein the Lactobacillus fermentum CQPC04 improves coagulation abnormality caused by thrombus.

4. The application of the Lactobacillus fermentum CQPC04 in preparing products for treating thrombus according to claim 1, wherein the Lactobacillus fermentum CQPC04 reduces oxidative damage and inflammatory responses caused by thrombus.


SEQUENCE LISTING (22 sequences)

SEQ ID NO: 1 - DNA, 21 bp, synthetic construct
  source 1..21: mol_type = other DNA
aaccagttgtgttgtgaggac

SEQ ID NO: 2 - DNA, 23 bp, synthetic construct
  source 1..23: mol_type = other DNA
ccaccatgtttcttagagtgagg

SEQ ID NO: 3 - DNA, 22 bp, synthetic construct
  source 1..22: mol_type = other DNA
cagacctgccttacgactatgg

SEQ ID NO: 4 - DNA, 21 bp, synthetic construct
  source 1..21: mol_type = other DNA
ctcggtggcgttgagattgtt

SEQ ID NO: 5 - DNA, 19 bp, synthetic construct
  source 1..19: mol_type = other DNA
ggaggcgggaacccaatag

SEQ ID NO: 6 - DNA, 21 bp, synthetic construct
  source 1..21: mol_type = other DNA
gtgtgccatctcgtcagtgaa

SEQ ID NO: 7 - DNA, 21 bp, synthetic construct
  source 1..21: mol_type = other DNA
gaggcacgaggctccttttct

SEQ ID NO: 8 - DNA, 23 bp, synthetic construct
  source 1..23: mol_type = other DNA
gtagctgcatggagactcgaaca

SEQ ID NO: 9 - DNA, 21 bp, synthetic construct
  source 1..21: mol_type = other DNA
tccgctaccatcaccgtgtat

SEQ ID NO: 10 - DNA, 20 bp, synthetic construct
  source 1..20: mol_type = other DNA
tagccagcaccgtgaatgtg

SEQ ID NO: 11 - DNA, 20 bp, synthetic construct
  source 1..20: mol_type = other DNA
ttgggagcctcaacggtact

SEQ ID NO: 12 - DNA, 23 bp, synthetic construct
  source 1..23: mol_type = other DNA
gcaatcgttttgtattcagggga

SEQ ID NO: 13 - DNA, 20 bp, synthetic construct
  source 1..20: mol_type = other DNA
ataacgagacgccatcatgc

SEQ ID NO: 14 - DNA, 18 bp, synthetic construct
  source 1..18: mol_type = other DNA
tgtccactgcccttgtgc

SEQ ID NO: 15 - DNA, 19 bp, synthetic construct
  source 1..19: mol_type = other DNA
atgaagttcctctctgcaa

SEQ ID NO: 16 - DNA, 19 bp, synthetic construct
  source 1..19: mol_type = other DNA
agtggtatcctctgtgaag

SEQ ID NO: 17 - DNA, 17 bp, synthetic construct
  source 1..17: mol_type = other DNA
atggggggcttccagaa

SEQ ID NO: 18 - DNA, 17 bp, synthetic construct
  source 1..17: mol_type = other DNA
cctttggggaccgatca

SEQ ID NO: 19 - DNA, 20 bp, synthetic construct
  source 1..20: mol_type = other DNA
gctttgcagctcttcctcat

SEQ ID NO: 20 - DNA, 20 bp, synthetic construct
  source 1..20: mol_type = other DNA
gtcaccatccttttgccagt

SEQ ID NO: 21 - DNA, 23 bp, synthetic construct
  source 1..23: mol_type = other DNA
tgacctcaactacatggtctaca

SEQ ID NO: 22 - DNA, 19 bp, synthetic construct
  source 1..19: mol_type = other DNA
cttcccattctcggccttg